(19) United States
(12) Patent Application Publication
(10) Pub. No.: US 2014/0007877 A1
(43) Pub. Date: Jan. 9, 2014

(54) CHILDBIRTH LABOR COACH WITH PACED BREATHING

(71) Applicant: Nathan Francis O'Connor, Monroeville, PA (US)

(72) Inventor: Nathan Francis O'Connor, Monroeville, PA (US)

(73) Assignee: KONINKLIJKE PHILIPS N.V., EINDHOVEN (NL)

(21) Appl. No.: 14/006,343

(22) PCT Filed: Mar. 13, 2012
(86) PCT No.: PCT/IB12/51184

§ 371 Date: Sep. 20, 2013

Related U.S. Application Data
(60) Provisional application No. 61/467,167, filed on Mar. 24, 2011.

(51) Int. Cl. A61M 16/00

(52) U.S. Cl. CPC A61M 16/0051; A61M 16/0057
USPC 128/204.23

(57) ABSTRACT

A system (10) configured to prompt a subject (12) to consciously alter one or more breathing parameters during childbirth. The system includes a pressure generator (14) that generates a pressurized flow of breathable gas for delivery to an airway of the subject during childbirth and a processor (22) that controls the pressure generator to adjust one or more gas parameters of the gas in the pressurized flow of breathable gas to provide breathing cues to the subject in accordance with a breathing regime associated with labor contractions, wherein the breathing cues prompt the subject to consciously alter one or more breathing parameters of respiration.

The present disclosure pertains to providing breathing cues to a subject, and in particular, in accordance with a breathing regime associated with labor contractions.

It is known that breathing regimes associated with labor contractions (e.g., Lamaze breathing) may be used by expectant mothers during childbirth to reduce pain and improve relaxation and comfort of the expectant mother. These breathing regimes are typically associated with the labor contractions. For example, as the pains of labor increases in intensity, the breathing regime may shift from a slow breathing pattern to a more rapid pattern, and then back to a slower breathing pattern. However, during labor, the expectant mothers may need assistance to help conform their breathing patterns to the breathing regime associated with labor contractions.

Accordingly, an aspect of one or more embodiments of the present disclosure to provide a system configured to prompt a subject to consciously alter one or more breathing parameters during childbirth. The system includes a pressure generator configured to generate a pressurized flow of breathable gas for delivery to an airway of the subject during childbirth. The system also includes a processor configured to control the pressure generator to adjust one or more gas parameters of the gas in the pressurized flow of breathable gas to provide breathing cues to the subject in accordance with a breathing regime associated with labor contractions. The breathing cues prompt the subject to consciously alter one or more breathing parameters of respiration.

It is yet another aspect of one or more embodiments of the present disclosure to provide a method of prompting a subject to consciously alter one or more breathing parameters during childbirth. The method includes the step of generating a pressurized flow of breathable gas for delivery to an airway of a subject during childbirth. The method also includes the step of controlling an adjustment to the one or more gas parameters of the gas in the pressurized flow of breathable gas in order to provide breathing cues to the subject based on a breathing regime associated with labor contractions. The breathing cues prompt the subject to consciously alter one or more breathing parameters of respiration.

It is yet another aspect of one or more embodiments of the present disclosure to provide a system configured to prompt a subject to consciously alter one or more breathing parameters during childbirth. The system includes means for generating a pressurized flow of breathable gas for delivery to an airway of a subject during childbirth. The system also includes means for controlling an adjustment to one or more gas parameters of the gas in the pressurized flow of breathable gas in order to provide breathing cues to the subject based on a breathing regime associated with labor contractions. The breathing cues prompt the subject to consciously alter one or more breathing parameters of respiration.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

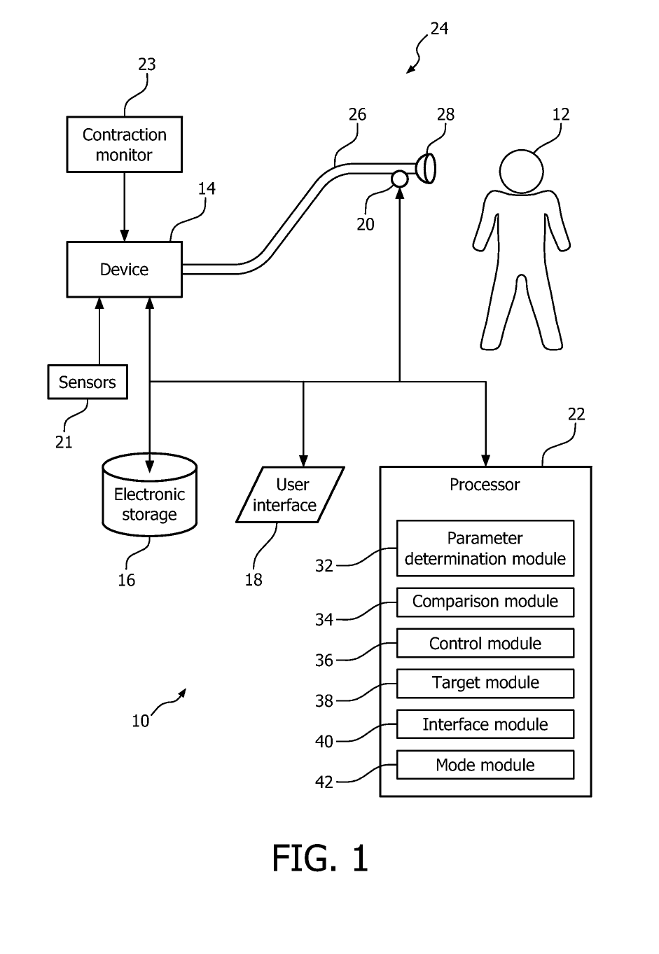

FIG. 1 illustrates a system configured to prompt a subject to consciously alter one or more breathing parameters during childbirth, in accordance with one or more embodiments of the invention;

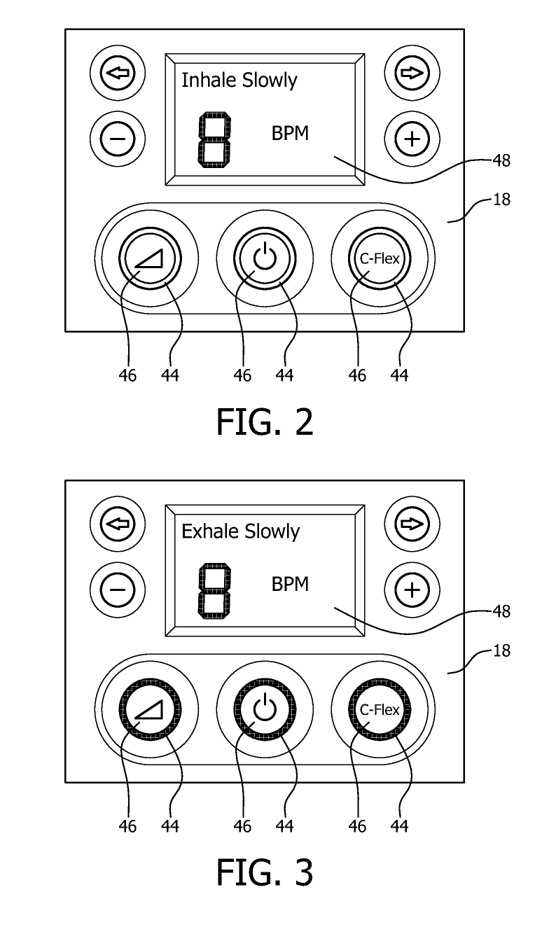

FIG. 2 illustrates a user interface configured to provide information to a subject related to breathing cues being provided to the subject in accordance with a breathing regime associated with labor contractions, according to one or more embodiments of the invention;

FIG. 3 illustrates a user interface configured to provide information to a subject related to breathing cues being provided to the subject in accordance with a breathing regime associated with labor contractions, according to one or more embodiments of the invention;

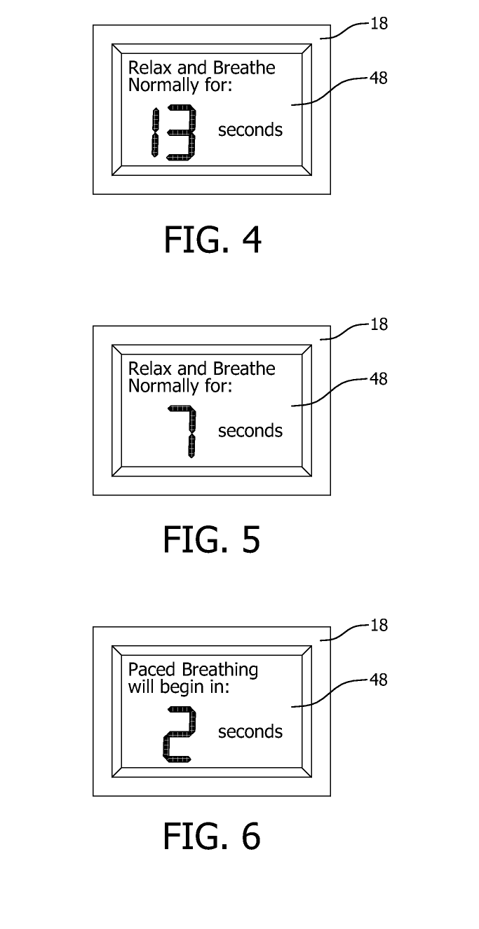

FIG. 4 illustrates a user interface configured to provide information to a subject related to breathing cues being provided to the subject in accordance with a breathing regime associated with labor contractions, according to one or more embodiments of the invention;

FIG. 5 illustrates a user interface configured to provide information to a subject related to breathing cues being provided to the subject in accordance with a breathing regime associated with labor contractions, according to one or more embodiments of the invention;

FIG. 6 illustrates a user interface configured to provide information to a subject related to breathing cues being provided to the subject in accordance with a breathing regime associated with labor contractions, according to one or more embodiments of the invention; and

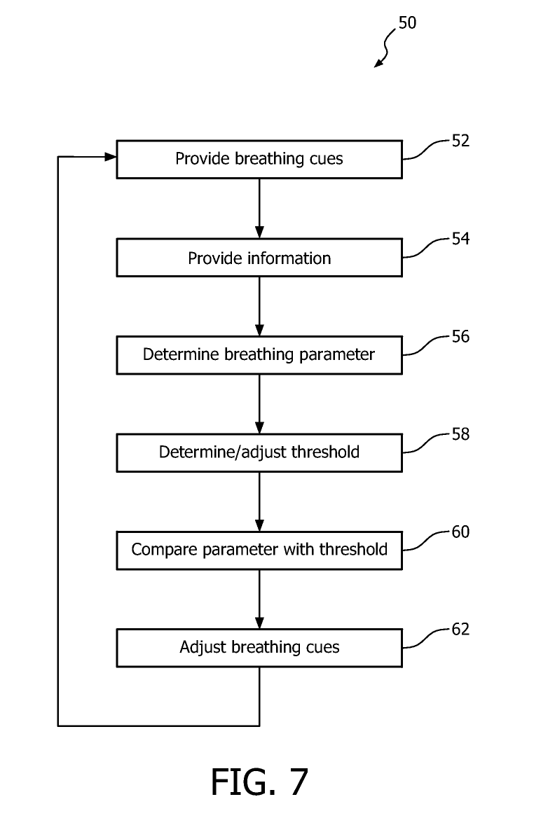

FIG. 7 illustrates a method of prompting a subject to consciously alter one or more breathing parameters during childbirth, according to one or more embodiments of the invention.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIG. 1 schematically illustrates an exemplary embodiment of a system 10 configured to prompt a subject 12 (i.e., an expectant mother) to consciously alter one or more breathing parameters during childbirth. To prompt subject 12 to alter one or more breathing parameters, system 10 provides pressurized flow of breathable gas to the airway of subject 12. System 10 adjusts one or more gas parameters of the gas in the pressurized flow of breathable gas to provide breathing cues to subject 12 that encourage subject 12 to consciously adjust respiration such that the one or more breathing parameters are altered to conform to a breathing regime associated with labor contractions (e.g., Lamaze breathing regime). System 10 may be further configured to provide information to subject 12 related to the breathing cues being delivered (or about to be delivered) by system 10 via the pressurized flow of breathable gas. This information may be provided to the user auditorily, visually, tactily, and/or via some other sensory feedback. The information related to the breathing cues may train subject 12 to understand the breathing cues delivered through the pressurized flow of breathable gas, to assume one or more therapeutic body positions, and/or serve other purposes. In one embodiment, system 10 may include a pressure generator device 14, electronic storage 16, a user interface 18, one or more gas parameter sensors 20, one or more physiological sensors 21, a contraction monitor 23, a processor 22, and/or other components.

In one embodiment, device 14 includes a positive pressure support device. A positive pressure support device is well-known and is disclosed, for example, in U.S. Pat. No. 6,105,575, hereby incorporated by reference in its entirety. In this embodiment, device 14 is configured to deliver a pressurized flow of breathable gas to the airway of subject 12.

Device 14 may be configured to generate the pressurized flow of breathable gas according to one or more modes. A non-limiting example of one such mode is Continuous Positive Airway Pressure (CPAP). CPAP has been used for many years and has proven to be helpful in promoting regular breathing. Another mode for generating the pressurized flow of breathable gas is Inspiratory Positive Air Pressure (IPAP). One example of the IPAP mode is bi-level positive air pressure (BiPAP). In BiPAP, two levels of positive air pressure (HI and LO) are supplied to a patient. Other modes of generating the pressurized flow of breathable gas are contemplated.

Generally, the timing of the HI and LO levels of pressure are controlled such that the HI level of positive air pressure is delivered to subject 12 during inhalation and the LO level of pressure is delivered to subject 12 during exhalation. In conventional positive pressure support devices, the timing of the HI and LO levels of pressure is coordinated to coincide with the breathing of subject 12 based on detection of gas parameters that indicate whether a user is currently inhaling or exhaling. The timing of the HI and LO segments of BiPAP may generate breathing cues to prompt subject 12 in changing her breathing rate. In some embodiments, the breathing cues may be provided using HI and LO levels as described in U.S. Pat. No. 7,556,038, which is incorporated herein in its entirety. In some embodiments, the breathing rate may be controlled by adjusting the tidal volume (air breathed per breath) delivered via controlled, progressive pressurization during inspiration and controlled de-pressurization during expiration.

It should be appreciated that device 14 may also deliver gases other than room air. For example, the device 14 can also be oxygen delivery devices, anesthesia devices, fresh air respirators, and respiratory (or other) drug delivery devices in other embodiments. It should also be appreciated that the cues may be positive pressure, negative pressure, or atmospheric.

The pressurized flow of breathable gas is delivered to the airway of subject 12 via a subject interface 24. Subject interface 24 is configured to communicate the pressurized flow of breathable gas generated by device 14 to the airway of subject 12. As such, subject interface 24 includes a conduit 26 and an interface appliance 28. Conduit conveys the pressurized flow of breathable gas to interface appliance 28, and interface appliance 28 delivers the pressurized flow of breathable gas to the airway of subject 12. Some examples of interface appliance 28 may include, for example, an endotracheal tube, a nasal cannula, a tracheotomy tube, a nasal mask, a nasal/oral mask, a full face mask, a total face mask, or other interface appliances that communication a flow of gas with an airway of a subject. The present invention is not limited to these examples, and contemplates delivery of the pressurized flow of breathable gas to subject 12 using any subject interface.

In one embodiment, electronic storage 16 comprises electronic storage media that electronically stores information. The electronically storage media of electronic storage 16 may include one or both of system storage that is provided integrally (i.e., substantially non-removable) with system 10 and/or removable storage that is removably connectable to system 10 via, for example, a port (e.g., a USB port, a firewire port, etc.) or a drive (e.g., a disk drive, etc.). Electronic storage 16 may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage 16 may store software algorithms, information determined by processor 22, information received via user interface 18, and/or other information that enables system 10 to function properly. Electronic storage 16 may be (in whole or in part) a separate component within system 10, or electronic storage 16 may be provided (in whole or in part) integrally with one or more other components of system 10 (e.g., device 14, user interface 18, processor 22, etc.).

User interface 18 is configured to provide an interface between system 10 and subject 12 through which subject 12 may provide information to and receive information from system 10. This enables data, results, and/or instructions and any other communicable items, collectively referred to as “information,” to be communicated between subject 12 and one or more of device 14, electronic storage 16, and/or processor 22. Examples of interface devices suitable for inclusion in user interface 18 include a keypad, buttons, switches, a keyboard, knobs, levers, a display screen, a touch screen, speakers, a microphone, an indicator light, an audible alarm, a printer, and/or other interface devices. In one embodiment, user interface 18 includes a plurality of separate interfaces. In one embodiment, user interface 18 includes at least one interface that is provided integrally with device 14.

It is to be understood that other communication techniques, either hardwired or wireless, are also contemplated by the present invention as user interface 18. For example, the present invention contemplates that user interface 18 may be integrated with a removable storage interface provided by electronic storage 16. In this example, information may be loaded into system 10 from removable storage (e.g., a smart card, a flash drive, a removable disk, etc.) that enables the user(s) to customize the implementation of system 10. Other exemplary input devices and techniques adapted for use with system 10 as user interface 18 include, but are not limited to, an RS-232 port, RF link, an IR link, modem (telephone, cable or other). In short, any technique for communicating information with system 10 is contemplated by the present invention as user interface 18.

One or more gas parameter sensors 20 are configured to generate one or more output signals conveying information related to one or more gas parameters of the gas breathed by subject 12. For example, the one or more gas parameter sensors 20 may be used to sense and convey information to determine the respiratory rate. The one or more gas parameter sensors 20 may also be configured to sense other parameters, such as, for example, one or more of a volume, a pressure, a composition (e.g., concentration(s) of one or more constituents), humidity, temperature, acceleration, velocity, acoustics, changes in a parameter indicative of respiration, and/or other gas parameters. In an embodiment in which a pressurized flow of breathable gas is delivered to subject 12 from device 14, sensors 20 include sensors in communication with gas within subject interface 24.

In addition, one or more optional additional physiological sensors 21 are configured to sense physiological characteristics of subject 12. For example, sensors 21 may include a pulse oximeter configured to monitor the oxygen saturation of a patient's blood. Sensors 21 may also include a cardiac monitor to monitor, for example, subject 12's cardiac rhythm and/or heart rate variability. It should be appreciated that the sensors 21 may also include other types of sensors and any combination and number thereof.

Contraction monitor 23 is configured to monitor labor contractions during childbirth. The contraction monitor 23 may be configured to monitor and communicate the frequency, magnitude, and pattern of contractions during labor. For example, the contraction monitor 23 may monitor and communicate the beginning of a contraction and the end of the contraction such that the length and pattern of contractions may be calculated. In one embodiment, the contraction monitor 23 may be part of a fetal monitor that also monitors the fetus's heart rate. The contraction monitor 23 may be any type of sensor that monitors the labor contractions and outputs signals associated with the labor contractions. Furthermore, the contraction monitor 23 may be external or internal and may communicate output signals via signal cables or wirelessly (e.g., IrDA, RFID (Radio Frequency Identification), Wireless USB). For example, the contraction monitor 23 may include pressure transducers or strain gauges held against subject 12's abdomen by an elastic belt placed around subject 12's waist. Contraction monitor 23 may be internal catheters inserted into the uterus to measure changes in the amniotic fluid pressure in the amniotic sac. Alternatively or additionally, the contraction monitor 23 may include a fiber optic strain sensor that generates signals in response to labor contractions and wirelessly communicates the output signals via a transceiver. Various attachment mechanisms may be used to attach the external contraction monitors 23 to subject 12, such as, just for example, an elastic band, a belt, or adhesive materials.

Processor 22 is configured to provide information processing capabilities in system 10. As such, processor 22 may include one or more of a digital processor, an analog processor, a digital circuit designed to process information, an analog circuit designed to process information, a state machine, and/or other mechanisms for electronically processing information. Although processor 22 is shown in FIG. 1 as a single entity, this is for illustrative purposes only. In some implementations, processor 22 may include a plurality of processing units. These processing units may be physically located within the same device, or processor 22 may represent processing functionality of a plurality of devices operating in coordination.

As is shown in FIG. 1, processor 22 may be configured to execute one or more computer program modules. The one or more computer program modules may include one or more of a parameter determination module 32, a comparison module 34, a control module 36, a target module 38, an interface module 40, a mode module 42, and/or other modules. Processor 22 may be configured to execute modules 32, 34, 36, 38, 40, and/or 42 by software; hardware; firmware; some combination of software, hardware, and/or firmware; and/or other mechanisms for configuring processing capabilities on processor 22.

It should be appreciated that although modules 32, 34, 36, 38, 40, and 42 are illustrated in FIG. 1 as being co-located within a single processing unit, in implementations in which processor 22 includes multiple processing units, one or more of modules 32, 34, 36, 38, 40, and/or 42 may be located remotely from the other modules. The description of the functionality provided by the different modules 32, 34, 36, 38, 40, and/or 42 described below is for illustrative purposes, and is not intended to be limiting, as any of modules 32, 34, 36, 38, 40, and/or 42 may provide more or less functionality than is described. For example, one or more of modules 32, 34, 36, 38, 40, and/or 42 may be eliminated, and some or all of its functionality may be provided by other ones of modules 32, 34, 36, 38, 40, and/or 42. As another example, processor 22 may be configured to execute one or more additional modules that may perform some or all of the functionality attributed below to one of modules 32, 34, 36, 38, 40, and/or 42.

The parameter determination module 32 is configured to determine one or more breathing parameters of the respiration of subject 12 from the one or more output signals generated by sensors 20. The one or more breathing parameters include the one or more breathing parameters that subject 12 is prompted to consciously alter by the breathing cues provided in the pressurized flow of breathable gas. The one or more breathing parameters may include breath rate, such as timing parameters of inhalation and/or exhalation (e.g., duration, frequency, relative length). The breathing parameters may also include one or more of an inhalation flow rate, an inhalation period, an exhalation flow rate, and/or an exhalation period. In some embodiments, breathing parameter module 32 may include a timer configured to determine the duration of inhalation and exhalation and/or the time therebetween to determine the breathing rate. In some embodiments, the one or more breathing parameters may optionally include parameters of the actual gas breathed by subject 12 (e.g., one or more of a tidal volume, a breath period, a peak flow, a flow curve shape, a pressure curve shape, and/or other breathing parameters).

Comparison module 34 is configured to compare the one or more breathing parameters determined by parameter determination module 32 to a target for the one or more breathing parameters that the breathing cues prompt subject 12 to consciously alter. For example, the target may be a target breathing rate. The target shape may be in accordance with breathing patterns associated with labor contractions, such as the Lamaze breathing pattern. As another example, if the breathing parameter is a curve shape, the target may include a target curve shape. The comparison module 34 may also receive information from the contraction monitor 23 and physiological sensors 21 during the comparison of the target with the breathing parameters determined by the parameter determination module 32. The target, which will be described in more detail below, may be based at least partially on information received from the contraction monitor 23 and physiological sensors 21.

Control module 36 is configured to control device 14. Controlling device 14 includes adjusting the breathing cues provided to subject 12 by device 14. As was mentioned above, in one embodiment, the breathing cues administered to subject 12 by device 14 include changes to one or more parameters of the pressurized flow of breathable gas delivered from device 14 to subject 12. In embodiments that use BiPAP therapy, the one or more parameters include changing the breathing rate by changing the timing, pattern or other variables associated with the application of HI and LO pressures. In some embodiments, the one or more parameters may also include a pressure, a flow rate, and/or a volume of the pressurized flow of breathable gas. Control module 36 may receive information from comparison module 34 to adjust the breathing cues provided to subject 12 via device 14 in order to prompt subject 12 to bring the one or more breathing parameters into conformance with the target associated with the breathing regime.

In an embodiment in which device 14 generates the pressurized flow of breathable gas according to a BiPAP mode, control module 36 may control device 14 to adjust the timing of the HI and LO segments. For example, when comparison module 34 determines that subject 12's breathing rate is greater than the target rate, control module 34 may control device 14 such that the time over which the HI pressure of the device 14 is supplied is increased and the time over which the LO pressure is supplied is adjusted. The LO pressure time may be adjusted in accordance with a fixed ratio between the HI and Lo pressure times, or may be adjusted to coincide with subject 12's actual expiration time. Thus, by controlling the pattern or timing of the application of HI and LO pressures, the breathing rate of subject 12 may be affected.

In other embodiments in which device 14 generates the pressurized flow of breathable gas according to a BiPAP mode, control module 36 may control device 14 to adjust other properties of the HI and LO pressures, such as a period of the HI and/or LO pressure cycles, a pressure curve shape during a transition between HI and LO pressure cycles, a flow rate curve shape during a transition between HI and LO pressure cycles, and/or adjust other gas parameters of the pressurized flow of breathable gas. Such adjustments to the gas parameters of the pressurized flow of breathable gas will tend to provide breathing cues to subject 12 to consciously alter other breathing parameters in addition to or instead of breath rate. For example, such breathing cues may also prompt subject 12 to alter one or more of a respiration flow curve shape, a respiration pressure curve shape, and/or other breathing parameters.

It is contemplated that in some embodiments, control module 36 may also control device 14 to adjust other breathing parameters of subject 12, such as the pressure, flow rate, and/or volume of gas delivered to the airway of subject 12 while the pressurized flow of breathable gas is being generated at the HI pressure (e.g, during inhalation) and/or at the LO pressure (e.g., during exhalation). Adjusting the pressure, flow rate, and/or volume of gas delivered to the airway of subject 12 while the pressurized flow of breathable gas is being generated at the HI pressure or at the LO pressure will tend to generate breathing cues that prompt subject 12 to alter the volume of gas inhaled/exhaled, to alter the inspiration/exhalation period, to alter the inspiration/expiration flow rate, to alter the tidal volume, and/or to otherwise consciously alter one or more other breathing parameters.

In one embodiment, adjustments to the parameters of the pressurized flow of breathable gas are made by control module 36 using feedback. In this embodiment, adjustments to the parameters of the pressurized flow of breathable gas may be determined based on the comparison between the breathing parameter and the target threshold made by comparison module 34. For example, if comparison module 34 determines that the gas parameters of the pressurized flow of breathable gas that are being adjusted to provide breathing cues to subject 12 are not adequate, control module 36 will adjust the gas parameters of the pressurized flow of breathable gas to provide more effective cues. Breathing cues would be identified as inadequate if comparison module 34 determines that the breathing cues are not successful in prompting subject 12 to consciously bring the one or more breathing parameters into conformance with the target for the one or more breathing parameters. That is, if subject 12 is not breathing in conformance with the breathing pattern associated with labor contractions, control module 36 may adjust the gas parameters of the pressurized flow of breathable gas to provide more effective cues. This adjustment may include adjustments for instances in which the conscious alteration of the one or more breathing parameters by subject 12 has not gone far enough (e.g., breathing is too close to normal breathing), and/or for instances in which the conscious alteration of the one or more breathing parameters by subject 12 has gone too far. For example, in embodiments using BiPAP therapy, if subject 12 is breathing at a different frequency or rate than the target frequency or rate, control module 36 may adjust the timing and pattern of application of the HI and LO pressures to bring subject 12's breathing parameter into conformance with the target.

In one embodiment, adjustments to the parameters of the pressurized flow of breathable gas are made without feedback. In this embodiment, relationships between the one or more gas parameters of the pressurized flow of breathable gas and the one or more breathing parameters to be consciously altered are determined in advance. These predetermined relationships are then used to generate the pressurized flow of breathable gas with gas parameters that correspond to the target for the one or more breathing parameters. The target may be based on a pre-determined breathing pattern associated with labor contractions. The target may be pre-programmed or manually input into system 10. In such embodiments wherein adjustments to the parameters are made without feedback, processor 22 may not include comparison module 34 and/or sensors 20.

Target module 38 is configured to obtain a target for the one or more breathing parameters to be consciously altered. In one embodiment, the target is received from a user (e.g., a caregiver, subject 12, etc.). The user may input the target via user interface 18. Inputting the target may include inputting a new target, or adjusting a previously obtained target. In one embodiment, subject 12 may input information via user interface 18 signaling the beginning, end, or duration of contractions. The target may be adjusted based on the duration of the contractions and the stage of the contraction. In such embodiments, the target breathing frequency or rate may be increased at the middle or potentially most intense portion of the contractions. Inputting the target may also include configuring the target from a predetermined template (e.g., corresponding to a certain breathing regime). Subject 12 or other users may also input information via user interface 18 to adjust or change the order of the breathing patterns and the target associated with the breathing patterns.

The target for the one or more breathing parameters corresponds to a breathing regime. For example, for a breathing regime associated with labor contractions that include a period of reduced breath rate, the target may include a target level for breath rate and/or one or more related breathing parameters. If the breathing regime includes a flow rate curve shape, the target may include a target curve shape. The target curve shape may be refined (e.g., by a user via user interface 18) to a target curve having values for the extrema (e.g., maxima and/or minima). The target may also be based on an algorithm used to predict the period and duration of contractions. For example, if the time period between contractions is decreasing, the algorithm may be used to predict the start of the next contraction based on the timing of past contractions so as to set an appropriate target based on the contractions. Subject 12 or other users may input information via user interface 18 to adjust the target or input further information for the predictive algorithm to set the target. In one embodiment, subject 12 may input information via user interface 18 at any time during therapy. Other targets corresponding to these and/or other breathing regimes may be implemented within the scope of this disclosure.

The target may be based on the output signals received from the contraction monitor 23 configured to monitor the labor contractions. The target may be associated with the breathing pattern that is dependent on the labor contractions. For example, during the beginning of the contraction, the breathing pattern may be slow. As the contraction intensifies, the breathing pattern may accelerate, and then may be slower towards the end of the contraction. The breathing pattern may then return to a normal respiratory rate between contractions. Accordingly, the target may be adjusted based on these breathing patterns. Similarly, the target may fluctuate over time during the presence or absence of contractions (e.g., little breaths then big breaths then little breaths during a contraction).

In one embodiment, target module 38 sets the target at an initial target, and then slowly modifies the target over time toward a final target. The initial target may be based on the baseline breathing parameters of subject 12, and/or may be preset (or preconfigured). Modifying the target over time from an initial target to a final target may enhance the comfort of the breathing cues provided to subject 12. Modifying the target over time may include incrementing the target, modifying the target over time smoothly, and/or otherwise modifying the target.

In one embodiment, target module 38 adjusts the target based on the one or more breathing parameters determined by breathing parameter module 32. For example, comparison module 34 may determine that subject 12 is not altering the one or more breathing parameters adequately to conform to the target. Based on this determination, target module 38 may adjust the target to make conformance easier, the breathing cues provided to subject 12 by device 14 may be adjusted by control module 36 to reflect the adjusted target. Target module 38 may then monitor the compliance of subject 12 with the new target (e.g., based on comparisons made by comparison module 34). If it is determined that subject 12 is complying with the new target, target module 38 will then adjust the target toward the previous target. If it is determined that subject 12 is not complying with the new target, then target module 38 will take a different action.

The breathing cues provided to subject 12 by manipulating one or more gas parameters of the pressurized flow of breathable gas may be an effective way to provide respiratory instruction to subject 12 to consciously alter one more breathing parameters. The conscious alteration of the one or more breathing parameters in response to the breathing cues may enable subject 12 to receive therapeutic benefits during the altered breathing, or to learn to consciously modify the one or more breathing parameters during periods when subject 12 is not connected to device 14. For example, subject 12 may learn breathing regimes effective for childbirth that can be executed (once learned) without the aid of system 10.

However, in some cases, subject 12 may initially have difficulty determining what the breathing cues being provided in the pressurized flow of breathable gas are prompting subject 12 to do. The interface module 40 is configured to dynamically (e.g., adjusted or updated based on the actual breathing cues) to provide information to subject 12 related to the meaning of the breathing cues provided to subject 12 by the pressurized flow of breathable gas generated by device 14. In one embodiment, interface module 40 controls user interface 18 to communicate the information related to the breathing cues to subject 12. The information related to the breathing cues may include, for example, instructions to begin exhaling, to end exhaling, to begin inhaling, to end inhaling, to breathe faster, to breathe slower, to pause respiration, and/or to otherwise consciously alter one or more breathing parameters.

The information related to the breathing cues may be provided to subject 12 by user interface 18 in the form of auditory signals, visual signals, tactile signals, and/or other sensory signals. By way of non-limiting example, user interface 18 may include a radiation source capable of emitting light. The radiation source may include, for example, one or more of at least one LED, at least one light bulb, a display screen, and/or other sources. The interface module 40 may control the radiation source to emit light in a manner that conveys to subject 12 information related to the breathing cues being provided to subject 12 by the pressurized flow of breathable gas. For instance, the radiation source may emit light when the breathing cues are prompting subject 12 to inhale, and may stop emitting light, or emit light of a different color, when the breathing cues are prompting subject 12 to exhale. The intensity of the light emitted by the radiation source may convey to subject 12 the magnitude of the flow that the breathing cues are prompting subject 12 to generate during respiration.

FIGS. 2 and 3 illustrate an embodiment in which interface module 40 controls a plurality of radiation sources 44 included in user interface 18 to emit radiation in a manner that provides information about the breathing cues being delivered by the pressurized flow of breathable gas. In particular, the plurality of radiation sources 44 are integrated with a set of buttons 46 disposed on device 14 to control device 14. In the embodiment illustrated in FIGS. 2 and 3, radiation sources 44 emit radiation when the breathing cues are prompting subject 12 to inhale, and stop emitting radiation when the breathing cues are prompting subject 12 to exhale.

Returning to FIG. 1, as another non-limiting example of the manner in which user interface 18 may communicate information about the breathing cues to subject 12, user interface 18 may include one or more elements capable of generating sounds that are audible to subject 12. The interface module 40 may control the element(s) to generate sounds that communicate to subject 12 the meaning of the cues being delivered to subject 12 by the pressurized flow of breathable gas. For instance, interface module 40 may control the element(s) to emit a “beep” or other short burst of noise to indicate to subject 12 a transition between inhalation and exhalation, and/or that flow should be increased or decreased. The interface module 40 may control the element(s) to play word messages that indicate to subject 12 the meaning of the breathing cues. The word messages may be prerecorded and stored within electronic storage 16.

As another non-limiting example of the manner in which user interface 18 may communicate information about the breathing cues to subject 12, user interface 18 may include one or more devices that contact subject 12 and provide tactile feedback to subject 12. For instance, user interface 18 may include a cuff that is worn by subject 12 around an extremity such as an arm, a leg, a finger, and/or other extremities. The cuff may carry one or more sensors configured to detect a physiological parameter of subject 12, such as for example, pulse, pulse rate, respiratory effort, blood pressure, blood oxygenation, and/or other physiological parameters. The cuff may vibrate and/or tighten on the extremity of subject 12 to provide information about the breathing cues to subject 12, such as a transition between inhalation and/or exhalation, or that flow should be increased or decreased.

As another non-limiting example of the manner in which user interface 18 may communicate information about the breathing cues to subject 12, user interface 18 may include a display screen that provides subject 12 with text conveying information about the breathing cues. The display screen may include, for instance, a screen provided on device 14 and/or other display screens. For instance, FIGS. 2 and 3 illustrate user interface 18 including a display screen 48 for conveying information to subject 12 about the breathing cues being delivered by the pressurized flow of breathable gas.

In one embodiment, interface module 40 controls user interface 18 to provide information about breathing cues that are currently being delivered to subject 12 and/or future breathing cues. By way of example, FIGS. 4-6 illustrate an embodiment of user interface 18 including display screen 48 in which interface module 40 controls display screen 48 to provide information to subject 12 regarding upcoming breathing cues.

Referring back to FIG. 1, mode module 42 is configured to manage the mode in which system 10 is operating. For example, mode module 42 may set learning modes in which interface module 40 controls user interface 18 to communicate information to subject 12 about the breathing cues being delivered through the pressurized flow of breathable gas, and normal modes in which information about the breathing cues is not provided to subject 12 through user interface 18. Mode module 42 may also set system 10 in feedback mode in which comparison module 34 is used to compare subject's 12 breathing rate with the target breathing rate, which may be based on information received from sensors 20, 21 and/or contraction monitor 23, and a non-feedback mode in which comparison module 34 is not used.

Interface module 40 may also be configured to provide visual focal cues for subject 12 via the user interface 18. The visual focal cues may include different color lights or displays of images, animations, or other visuals. Subject 12 may focus on these visual focal cues during labor. Interface module 40 may also provide relaxing music or other sounds (e.g., sounds associated with nature or other calming sounds) via user interface 18. In some embodiments, interface module 40 may also provide aromatherapy through user interface 18. Alternatively or additionally, interface module 40 may also be configured to provide messages of encouragement via user interface 18. The timing or pattern of these focal cues, music, messages, or aromatherapy may be determined based on information received from sensors 20, contraction monitor 23, and/or physiological sensors 21.

In some embodiments, interface module 40 may be configured to provide recommended body positions for subject 12 via user interface 18. Changing body positions at an interval (e.g., 30 minutes or any other time periods) between contractions may improve the comfort of subject 12. Information received from contraction monitor 23, sensors 20, and/or physiological sensors 21 may be used to determine the timing of the recommendations to change body positions. For example, information received from contraction monitor 23 may be used to determine the time period between contractions when the recommendations should be provided.

The mode module 42 may be configured to enable subject 12 to manually switch between a learning mode and a normal mode. This would enable subject 12 to selectively disable the provision of information to subject 12 through user interface 18 about the breathing cues. Inputs to mode module 42 to select a mode of operation for system 10 may be accomplished by subject 12 (or some other user) via user interface 18.

FIG. 7 illustrates a method 50 for prompting a subject to consciously alter one or more breathing parameters during childbirth. The operations of method 50 are intended to be illustrative. In some embodiments, method 50 may be accomplished with one or more additional operations not described, and/or without one or more of the operations discussed. Additionally, the order in which the operations of method 50 are illustrated in FIG. 7 and described below is not intended to be limiting. In some embodiments, method 50 may be implemented in a system that is similar to or the same as system 10 (shown in FIG. 1 and described above).

At an operation 52, breathing cues are provided to the self-ventilating subject. The breathing cues prompt the subject to breathe such that the breathing rate of subject 12 is in compliance with the target breathing rate that is based on a breathing regime associated with labor contractions. In one embodiment, the breathing cues include changes in one or more parameters of a pressurized flow of breathable gas being delivered to the airway of the subject. In one embodiment that uses BiPAP therapy, the changes include changes in patterns or timing of the application of HI and LO pressures. In one embodiment, the breathing cues are provided by a device that is the same as or similar to device 14 (shown in FIG. 1 and described above). The device may be controlled by a control module that is the same as or similar to control module 36 (shown in FIG. 1 and described above).

At operation 54, information relating to the meaning of the breathing cues are provided to subject 12. As mentioned above, the information related to the breathing cues may include, for example, instructions to begin exhaling, to end exhaling, to begin inhaling, to end inhaling, and other information. In operation 54, other information may also be provided to subject 12, such as a recommended body position based on the stage of contractions or timing between contractions sensed by contraction monitor 23 or based on information entered manually via user interface 18. Alternatively or additionally, visual focal cues or aromatherapy may also be provided during operation 54. In one embodiment, operation 54 is performed by an interface module similar to or the same as interface module 40 (shown in FIG. 1 and described above).

At an operation 56, a breathing parameter of the subject is determined. The breathing parameter is the breathing rate of subject 12, which may include a timing and/or duration of inhalation and/or exhalation. In one embodiment, operation 56 is performed by a parameter determination module that is the same as or similar to parameter determination module 32 (shown in FIG. 1 and described above).

At an operation 58, the target threshold is adjusted based on feedback information received from sensors 20, 21. The target may be adjusted based on the stage, duration, and other properties of contraction. The stage, duration, or other properties of contraction may be sensed by contraction monitor 23, predicted by an algorithm as discussed above, or may be manually inputted via user interface 18. Alternatively or additionally, the target may also be adjusted based on other information manually inputted via user interface 18. In one embodiment, the setting and adjustment of the target threshold may be performed by a target module that is the same as or similar to target module 38 (shown in FIG. 1 and described above).

At an operation 60, the breathing parameter determined at operation 54 is compared with a target threshold. The target threshold is, or corresponds to, a target breath rate in accordance with a breathing regime associated with labor contractions (e.g., Lamaze breathing regime). In one embodiment, operation 60 is performed by a comparison module that is the same as or similar to comparison module 34.

At an operation 62, the breathing cues provided to the subject are adjusted. The adjustment to the breathing cues is determined based on the comparison performed at operation 60. In one embodiment, operation 62 is performed by a control module that is similar to or the same as control module 36 (shown in FIG. 1 and described above).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A system configured to prompt a subject to consciously alter one or more breathing parameters during childbirth, the system comprising:
a pressure generator configured to generate a pressurized flow of breathable gas for delivery to an airway of the subject during childbirth; and
a processor configured to control the pressure generator to adjust one or more gas parameters of the gas in the pressurized flow of breathable gas to provide breathing cues to the subject in accordance with a breathing regime associated with labor contractions, wherein the processor is configured to receive information about the labor contractions from a contraction monitor (23) configured to monitor the labor contractions and generate output signals related to the labor contractions, and wherein the breathing cues prompt the subject to consciously alter one or more breathing parameters of respiration.

2. The system of claim 1, further comprising a contraction monitor, wherein the processor is configured to control the pressure generator to adjust the one or more gas parameters responsive to at least the output signals generated by the contraction monitor indicating the labor contractions.

3. The system of claim 1, further comprising a user interface configured to communicate with the subject, wherein the processor is further configured to control the user interface such that the user interface communicates information to the subject related to the meaning of the breathing cues provided to the subject by the pressurized flow of breathable gas generated by the device.

4. The system of claim 3, wherein the processor is further configured to control the user interface such that the user interface communicates information related to a recommended body position of the subject.

5. The system of claim 1, wherein the process is further configured to adjust the one or more gas parameters according to a predetermined target associated with the breathing regime.

6. A method of controlling a pressure generator to prompt a subject to consciously alter one or more breathing parameters during childbirth, the method comprising:
generating a pressurized flow of breathable gas for delivery to an airway of the subject during childbirth;
controlling an adjustment to the one or more gas parameters of the gas in the pressurized flow of breathable gas in order to provide breathing cues to the subject based on a breathing regime associated with labor contractions, wherein information related to the labor contractions is received from a contraction monitor configured to monitor the labor contractions and generate output signals related to the labor contractions, and wherein the breathing cues prompt the subject to consciously alter one or more breathing parameters of respiration.

7. The method of claim 6, further comprising monitoring labor contractions and generating output signals responsive to the labor contractions, wherein the one or more gas parameters are adjusted responsive to at least the output signals indicating the labor contractions.

8. The method of claim 6, further comprising communicating information to the subject related to the meaning of the breathing cues provided to the subject by the pressurized flow of breathable gas, wherein the information is communicated to the subject dynamically with the provision of the breathing cues.

9. The method of claim 8, further comprising communicating information related to a recommended body position of the subject.

10. The method of claim 6, wherein the one or more gas parameters are adjusted according to a predetermined target associated with the breathing regime.

11. A system configured to prompt a subject to consciously alter one or more breathing parameters during childbirth, the system comprising:
means for generating a pressurized flow of breathable gas for delivery to an airway of a subject during childbirth;
means for controlling an adjustment to one or more gas parameters of the gas in the pressurized flow of breathable gas in order to provide breathing cues to the subject based on a breathing regime associated with labor contractions, wherein the means for controlling is configured to receive information about the labor contractions from means for monitoring labor contractions (23) and generating output signals related to the labor contractions, and wherein the breathing cues prompt the subject to consciously alter one or more breathing parameters of respiration.

12. The system of claim 11, further comprising means for monitoring labor contractions and generating output related to the labor contractions, wherein the means for controlling is further configured to control the adjustment to the one or more gas parameters responsive to at least the output signals generated by the means for monitoring labor contractions indicating the labor contractions.

13. The system of claim 12, further comprising means for communicating information to the subject related to the meaning of the breathing cues provided to the subject by the pressurized flow of breathable gas, wherein the information is communicated to the subject dynamically with the provision of the breathing cues.

14. The system of claim 13, wherein the means for communicating is further configured to communicate information related to a recommended body position of the subject.

15. The system of claim 11, wherein the means for controlling is further configured to control the adjustment to the one or more gas parameters according to a predetermined target associated with the breathing regime.
